(19) United States
(12) Patent Application Publication
(10) Pub. No.: US 2003/0162232 A1
(43) Pub. Date: Aug. 28, 2003

(54) Component comprising a plurality of fiber elements and sample molecules that are immobilized on said fiber elements

(76) Inventor: Derya Ozkan, Berlin (DE)

Correspondence Address: MCGLEW & TUTTLE, PC, SCARBOROUGH STATION, SCARBOROUGH, NY, 10510, US

(21) Appl. No.: 10/220,334

(22) PCT Filed: Feb. 27, 2001
(86) PCT No.: PCT/DE01/00813

(51) Int. Cl. G01N 33/53; G01N 33/542; C12M 1/34

(52) U.S. Cl. 435/7.9; 435/287.2

(57) ABSTRACT

The invention relates to a component comprising a plurality of fiber elements and sample molecules of a selected sample molecule species or of selected sample molecule species groups, said sample molecules being immobilized on said fiber elements, whereby a specific sample molecule species or sample molecule species group is assigned to each fiber element. The invention is characterized in that the sample molecules are immobilized on outer surfaces of the fiber elements, and in that a supporting element fixes the fiber elements in an interspaced manner and in a radial direction with regard to the fiber elements or the fiber elements are bundled together with linear contact.

### FIELD OF THE INVENTION

The invention relates to a component comprising a plurality of fiber elements and sample molecules of selected sample molecule species or selected sample molecule species groups that are immobilized on said fiber elements, to each fiber element being associated at least one specific sample molecule species or sample molecule species group, to a method for producing such a component and to the use of such a component.

### BACKGROUND OF THE INVENTION AND PRIOR ART.

Components of the type referred to above serve for a quick analysis of samples for the presence or absence of target molecules. Basically it is a parallel method, since a sample is contacted simultaneously with several or all elements of the component and the target molecules react with those elements that carry sample molecules being specific for the target molecule.

Biochips are known in various embodiments. For instance the document U.S. Pat. No. 5,744,305 describes a biochip with a planar structure, on the surface of which at defined regions, so-called spots, respectively selected and associated sample molecules are applied. Such planar biochips are very costly in production, since every spot has to be provided sequentially with the respectively associated sample molecules. Furthermore, every component is so to speak unique, i.e. the production of a second biochip of the same structure, also same arrangement of sample molecules, will again require the same high costs as the production of the first biochip. A mass production is thus not possible, and the in so far known biochips are therefore extremely expensive.

A component of a different structure is known from the document U.S. Pat. No. 6,037,186. According thereto, a plurality of porous rods are produced, which are so to speak soaked with a solution containing a selected sample molecule species. After bundling the soaked rods, discs are cut off from the bundle in a plane orthogonally to the longitudinal extension of the bundle, said discs forming the component. The cutting faces are the spots. This technology does permit a mass production of a biochip, has however the enormous disadvantage that contaminations of the spots with the sample molecules of respectively adjacent spots, and as a consequence to a disturbing extent “crosstalking” being present during reading-out. Various conventional detection methods can furthermore not be used anymore for porous bodies. Finally, porous bodies have poorer kinetic properties due to diffusion-controlled transport processes in the interior of the pores.

Both components described above have the common drawback that only a small effective sensor area with sample molecules is available. Further, for reading-out, a suitable detector must precisely be positioned above the surface, however not making contact with the latter. With increasing miniaturization, the positioning problems will grow, and the risk of faulty associations between signals and spots will become considerably higher.

A component of the species referred to hereabove is known in the art from the document U.S. Pat. No. 5,837,196. In the in so far known component there is provided a bundle of optical fiber elements, the front faces of which arranged in one plane carrying the sample molecules. Reading-out is achieved by evaluation of optical signals at the opposite end of the fiber elements carrying the sample molecules. With this structure, the problems of the positioning of the detector are eliminated, but for the production of a component operating as a biochip, every time the produced front faces have to be provided with sample molecules with the result that an economic mass production is not possible, same as in the case of a biochip according to the above document U.S. Pat. No. 5,744,305.

Technical Object of the Invention.

The invention is based on the technical object to specify a component with immobilized sample molecules that can easily be produced in mass production and that simultaneously can safely be read out.

Basics of the Invention.

For achieving this technical object, the invention teaches that the sample molecules are immobilized on outer surfaces of the fiber elements, and that the fiber elements are fixed by means of a supporting element in a radial direction with regard to the fiber elements in an interspersed manner or are bundled together with linear contact. It cannot be excluded that sample molecules are also bound in the volume of the fiber elements, it is however essential that the interaction with target molecules takes place at or by means of the outer surfaces.

Further the invention teaches a method for the production of a component according to the invention, comprising the following steps: a) at least one endless fiber is produced, b) the endless fiber is guided through a fluid containing a selected sample molecule species or a selected sample molecule species group, c) the sample molecules of the sample molecule species or sample molecule species group are immobilized on the endless fiber, d) as an option the endless fiber is supplied to at least one washing step, e) to the endless fiber is directly or indirectly associated the sample molecule species or sample molecule species group immobilized on the fiber in step c), f) from different endless fibers or from different regions of an endless fiber, one fiber element each is cut off, and the fiber elements are connected or bundled with a supporting element. In principle, a plurality of endless fibers can each be guided through different fluids containing sample molecule species or sample molecule species groups. Alternatively, for a single endless fiber, the fluid is changed section-wise.

Finally, the invention teaches the use of a component according to the invention in a method for the detection of target molecules, wherein optically contactable front faces of the fiber elements are optically connected for instance to a CCD array or by a micro-mirror system to a photomultiplier being sensitive to optical radiation of the wavelength to be detected, and wherein sensor elements of the CCD array or micro-mirror or micro-mirror positions are each associated to the fiber elements, comprising the following steps: a) to the component is supplied a solution with prospective target molecules, under conditions at which target molecules bind to sample molecules, b) simultaneously with step a) or subsequently thereto the component is irradiated with a primary radiation exciting a wavelength to be detected, c) simultaneously with step b) or subsequently thereto a reading-out of the signals of the sensor elements of the CCD array or of the photomultiplier and processing and storage of the signals is performed. Alternatively or additionally, the fiber elements can for instance be electrically connectable and/or contacted, for the purpose of the evaluation by measurement of the impedance or impedance changes. Evaluations by detection of surface plasmon resonances or scattering processes are also possible. Above all, luminescence detection is also possible. For the purpose of the invention, the term binding also comprises interactions in the broadest sense.

Definitions.

A component is a device carrying sample molecules of a sample molecule species or sample molecule species group in discrete and defined surface regions. Normally every surface region of a component will carry a different sample molecule species or sample molecule species group. The surface regions are addressable in the sense that an association is/has been made between every surface region or its geometric position in the biochip and the sample molecule species or sample molecule species group carried by the surface region.

The term component also comprises the terms of the biochip and of the “composed analysis system from a plurality of independent individual elements”.

As fiber elements are designated pieces cut off from an endless fiber. Normally, the cut will be made in a plane orthogonally to the longitudinal extension of the endless fiber, however, of course, a cutting plane at an angle less than 90° is also possible.

An endless fiber is a rod-type or thread-type structure having a large longitudinal extension compared to the length of the fiber elements, typically produced by means of drawing technologies, blowing technologies and/or extrusion technologies and wound up and stored on drums or the like.

An endless fiber and/or a fiber element may comprise in a cross-sectional plane orthogonally to the longitudinal extension the most various cross-sectional shapes. Just preferred is a substantially circular cross-section. In so far the term outer surface comprises, for the purpose of the invention, not only cylindrical outer surfaces, but also outer surfaces in the case of non-circular cross-sections. In so far the term of the radial direction designates, for the purpose of the invention, all directions in a cross-sectional plane. In so far finally the diameter, for the purpose of the invention, is d=2·(F/2¶)^{0.5}, F being the cross-sectional area (any shape).

The front face of a fiber element is formed by a cut through an endless fiber.

Spacing of the fiber elements means that the outer surfaces of adjacent fiber elements do not touch each other. Then an bundling of the fiber elements without linear contact between individual fiber elements of the bundle has been achieved. Linear contact means that the (mechanical) contact does not exist in regions of mutually parallel faces of adjacent fiber elements. Equivalent to a spacing of the fiber elements and/or a linear contact between adjacent fiber elements is the provision of noses extending in a radial direction in the region of the outer surface of a fiber element, thus adjacent fiber elements being held spaced to each other except for the point, linear or areal contact in the region of the noses.

A fiber element bundle usually comprises mutually coplanar front faces of the bundled fiber elements. It is however also possible to adapt within a fiber element bundle groups of fiber elements with respectively coplanar front faces, the front faces of fiber elements of different groups not being mutually coplanar.

Optical fiber elements are optically transparent for electromagnetic radiation, at least in a partial section of the ranges IR, visible light and/or UV. Optically transparent means that the attenuation of the electromagnetic radiation is sufficiently low in order to permit a detection of electromagnetic radiation produced at one end of a fiber element at the opposite end of the fiber element by means of usual detection technologies.

The term optical contactability designates a treatment of a partial face of a fiber element permitting the emission of electromagnetic radiation out of the fiber element through the partial face. A strong scattering should be prevented, if possible. It could be of help to machine the partial faces in a suitable way, for instanced smooth or polish. Slight polishing or applying micro-lenses for focussing the emitted radiation is also possible.

Sample molecules are molecules that can enter a specific interaction with target molecules. Examples for such interactions are: antibody-antigen, lectin-carbohydrate, protein-aptamer, nucleic acid-nucleic acid, nucleic acid-ribozyme, biotin-avidin, etc.

Target molecules are molecules for which a sample to be analyzed and supplied to the component is examined. Target molecules may however also be molecules that specifically are to be removed from a sample (to be analyzed by other methods or by the same method).

A sample molecule species includes sample molecules with exclusively one structure, for instance of a sequence in the case of nuclear acids or proteins or peptides.

A sample molecule species group includes as group elements at least two sample molecule species. Sample molecule species may be identical or different sample molecule types. As sample molecule types are designated for instance nucleic acids, peptides, proteins and saccharides.

Functional groups of a polymeric material are such chemical groups of the polymeric structure that permit an unspecific binding between target molecules and the polymeric material. In the case of nucleic acids as target molecules, functional groups would be for instance amino groups, hydroxyl groups, thiol groups or carboxyl groups. It is understood that the above also applies to any auxiliary substances possibly added to the polymeric material.

Co-operative effects between molecules of several sample molecule species and a target molecule species are characterized by that the energy gain by simultaneous interaction between respectively the molecules of the different sample molecule species on one hand and that between the different sample molecule species and the target molecule as a whole on the other hand is greater than the sum of the energy gains of the interactions of a molecule of respectively one sample molecule species with one molecule of the target molecule species. In the case of the nucleic acids as sample molecules and target molecules are for instance to be named stacking effects. The stacking effect is an energy gain by interactions, namely delocalization of the ¶ electrons of the hydrophobic ring structures of adjacent bases in double-stranded nucleic acids. In the case of the proteins, co-operative effects may result from special secondary structures of proteins interacting with each other. Generally, a higher specificity and binding energy of a binding between sample molecules and a target molecule is achieved with co-operative effects.

### PREFERRED EMBODIMENTS OF THE INVENTION

In principle, the fiber elements may have any shapes in the direction of the longitudinal extension. With regard to a safe, spaced fixing over the complete longitudinal extension of the fiber elements, it is recommended to arrange the fiber elements in a straight line and possibly parallely or with a spacing to each other that grows in the longitudinal extension. It is understood that the spacing of the fiber elements is possibly so adapted that under consideration of potential lateral mechanical load forces of the fiber elements and of the modulus of elasticity of the fiber material as well as of the length thereof, the outer surfaces of adjacent fiber elements will not come into contact with each other, under normal operating conditions of the biochip. The alternatives already mentioned above will however also apply.

It is preferred that the fiber elements are optical fiber elements. With this embodiment, signals, for instance fluorescence signals from the corresponding marker groups of molecules bound to the fibers can (optically) be excited and guided to the detection by means of optical sensors to optically contactable locations of the fiber elements. Luminescence can however also be detected. Such locations may in particular be the front faces of the fiber elements. Optical fibers may for instance at least partially be made from glass. It is however preferred that the fiber elements are made from a polymeric material, preferably selected from the group consisting of “polyethylene (PE), polycarbonate (PC), polyvinyl chloride (PVC), polystyrene (PS), polyethylene terephthalate (PETP), polyethersulfone (PES), polyetherether ketone (PEEK), polyphenylene oxide (PPO), polyphenylene sulfide (PPS), polybutylene terephthalate (PBT), polyoxymethylene (POM), polysulfone (PSU), polyetherimide (PEI), polyamide (PA) and mixtures and copolymers of the monomers of such polymers”, in particular selected from the group consisting of “polycarbonate (PC), polyvinylchloride (PVC), polystyrene and mixtures and copolymers of the monomers of such polymers”. It is only essential when selecting the material that the material is sufficiently (permanently) resistant against the temperatures occurring during processing or use of the biochips. As in this meaning highly temperature-stable are designated all polymeric materials that prove to be stable under exposure of at least 90° C., preferably at least 120° C., in particular at least 140° C. over 1,000 hours. Typically this is achieved, if the glass temperature is above the mentioned temperature limit. Particularly temperature-stable is for instance polycarbonate with a glass temperature of 150° C. It is understood that the polymeric material may contain auxiliary substances that are common in plastics technology, such as softeners, light stabilizers, in particular UV stabilizers, and the like. Further, additions may be provided affecting the (wavelength-dependent) dielectric constant for the purpose of optimizing the optical properties in an interesting wavelength range.

A fiber element may also be made from several materials in combination. In particular in the region of the outer surface, materials different from the core material may be used, for instance in order to modify the reflectivity of light passing through the fiber element at the border face solid/liquid or solid/gaseous, possibly in dependence of the wavelength. Further, the core may for instance consist of a mechanically rigid material, for instance metal, glass or polycarbonate, whereas the optically transparent material around the core may then be less rigid.

With regard to geometry, the fiber elements may be adapted and arranged as follows. The fiber elements preferably have a diameter in the range from 0.01 μm to 1,000 μm and a length in the range from 0.1 μm to 100 mm. The ratio diameter to length may be in the range from 100 to 10⁻⁴. Further it is preferred that the fiber elements are packed in a density of 1 to 10⁷ fibers/cm², referred to a radial cross-section plane of the fiber elements.

With regard to the supporting element, various embodiments are possible. The supporting element may be arranged at one end of the fiber element and structured so to enclose the ends of the fiber elements, the front faces of the enclosed fiber elements being directly or indirectly optically contactable. In the case of the indirect contactability, the supporting element must be optically transparent, at least in the region of the ends of the fiber. Such a supporting element is typically plate-shaped, and its main faces are orthogonal to the longitudinal extension or middle axis of the fiber elements. Such a component can for instance be made by that the supporting element adapted as a perforated plate is completed with the fiber elements by introduction of the fiber elements or of the endless fiber into the holes of the perforated plate and subsequent fixing of the fiber elements in the holes. In the case of the introduction of endless fibers, a cutting step has of course to be performed prior to or after fixing. It is also possible to dip the ends of a bundle of fiber elements (or ends of endless fibers) held by means of a holding device into a not hardened material and perform then the hardening. As a material for such supporting elements, in principle the same materials can be used as described above in conjunction with the fiber elements. It is however recommendable to make the supporting element optically not transparent, for instance by adding pigments, and to adapt the fiber elements fully passing through the supporting element. Thereby, crosstalking of optical signals is reduced.

The supporting element may also be configured as a wound-up supporting ribbon. This is a long, ribbon-type elastic construct, for instance of a thermoplastic elastomer, such as thermoplastic polyurethane (TPU), at or in which one end of the fiber elements is applied or embedded. Therein the fiber elements are arranged orthogonally to the longitudinal extension of the wound-up supporting ribbon. Then the wound-up supporting ribbon is for instance spirally rolled up or folded in a meander-type zigzag manner, the fiber elements being arranged so to form a 2-dimensional pattern.

Further, one or more supporting elements may be formed from the fiber elements, for instance by contact-fixing in one or more regions of the fiber elements and melting, welding, gluing or the like of these regions of the fiber elements. The fiber elements may then extend in a parallel orientation and in linear contact with each other; if however during jointing of the regions, a mechanical pressure is exerted on the regions in a radial direction, non-parallel and contactless orientations of the fiber element may also be generated.

A component may comprise one supporting element only. This supporting element may in principle be arranged at every position referred to the longitudinal extension of the fiber elements, for instance at one end or in the center of the fiber elements. It is equally possible to have two supporting elements for a biochip. This is recommendable, in particular, in the case of very long and/or flexible fiber elements. If several supporting elements are provided, it is further recommendable to provide at any case two supporting elements each at the opposite ends of the fiber elements.

The sample molecule species or sample molecule species group may be selected from the group consisting of “nucleic acids, DNA, RNA, PNA, aptamers, proteins, peptides, saccharides and mixtures of these sample molecules”. In the case of nucleic acids, these may be single-stranded or double-stranded nucleic acids. The bases of the nucleic acids may be naturally existing bases, they may however also be chemically derivatized, for instance by integration of marker groups. Corresponding considerations apply in the case of the further compound classes of the above group. Further, the sequences may be natural or non-natural. In the case of the nucleic acids, the number of the bases of a hybridizable region may in principle be any whatever. It is however recommendable to keep the number of the bases as small as possible, for instance limit it to 25 at most, better 17, in order that a mismatch between a sample molecule and a target molecule in only one base will lead to a sufficient destabilization.

The sample molecules may be bound immediately or by spacer compounds to the fiber elements. The latter is preferred. As spacer compounds may be used any conventional suitable compound. For instance, a spacer compound may be constituted from a chain (for instance of the length 5 to 80, preferably 25 to 40 bases) of (identical) nucleic acid bases, for instance thymidine. Then it is advantageous if the number of the bases in the spacer compound is greater than the number of the bases in a hybridizable region of the sample molecule. It is also possible to use synthetic organic oligomers or polymers, in the polymer chain of which for instance carbamate groups may be integrated. It is also possible to connect two or more sample molecules by a spacer compound, and to bind the spacer compound by a binding site of the spacer compound to the fiber element.

Every fiber element may carry sample molecules of a respectively selected different sample molecule species. In other words: every fiber element carries sample molecules of a single structure, the structures of the sample molecules of different fiber elements being different. It is however also possible that every fiber element carries sample molecules of a respectively selected different sample molecule species group, the group elements of every sample molecule species group commonly binding under generation of co-operative effects, for instance stacking, to a defined target molecule. Then every fiber element carries sample molecules with two (or more) different structures (group elements), different fiber elements carrying different combinations of sample molecules of different structures. With regard to the used co-operative effects it is recommendable to keep the molar quantities of the sample molecules of different structures on a fiber element equal within deviations of-up to ±50%. It is recommendable that the sample molecules of different structures should be stochastically distributed on the fiber element, with regard to the spatial arrangement of the binding sites.

It is also possible to arrange within one or more of the fiber elements discrete fields carrying different sample molecule species or sample molecule species groups.

With regard to a reduction of unspecific binding of target molecules directly to the surfaces of the fiber elements, it is advantageous if the polymeric material does not carry any functional groups at its surface, and if the sample molecules are nucleic acids, that the nucleic acids from a preferably aqueous solution are bound under irradiation with UV light to the surface of the polymeric material.

For nucleic acids and other substance groups, a combinatorial synthesis on the fibers is possible. Of course, a component according to the invention may not only be used analytically, but also preparatively, for instance for the specific separation of substances from complex mixtures, such as blood. A component according to the invention may be provided, for instance by means of detergents, with a self-wetting surface in the region of the outer surfaces. Then solutions with target molecules will automatically wet the outer surfaces. It is also possible to form integrated devices, by means of a combination of components for instance for the sample preparation, for the analysis and reading-out. For the purpose of the production of the endless fibers and/or the fiber elements, the polymerization or structuring of the polymeric material may also be formed in presence or under addition of the sample molecules, so to integrate the sample molecules in the volume, which however are only accessible at the surface of a fiber element. Further the generation of cartridges containing at least one component according to the invention is possible, such cartridges being combinable for instance in series. On the surface of the components or in a cartridge, auxiliary substances, such as enzymes for PCR or ligation, but also interaction-mediating molecules may be located.

A component according to the invention may be integrated and fixed in a fluidic component, for instance a pipette tip or nozzle. Several components according to the invention may be connected fluidically in series or in parallel. An incorporation in integrated analysis systems is possible.

For components using stacking effects, Foerster energy transfer (FRET) can be applied, by that one of the two immobilized oligonucleotides is marked with a donor fluorescence dye and the other one is marked with an acceptor dye. During stacking then a Foerster transfer takes place, corresponding to a modulation of the emission wavelength. A modification of the electric conductivity for instance for the impedance measurement may also be performed.

Furthermore, a detection by means of solid, possibly metallic particles bound to target molecules is possible. Then a measurement is for instance made by scattering, evanescent waves or by metallic deposition at a fiber element (for instance, if silver nanoballs are bound to target molecules and photographic emulsions are used). In conjunction with these methods, it may also be possible to use SERS (surface enhanced Raman spectroscopy).

Due to the fact that the component is composed of different individual elements, every single element can be addressed. Complete spectra can be taken, and a more accurate statement with regard to the composition of the specific binding partners or even molecules freely existing in the solution can be made. For this can for instance be used: Raman, SERS, NIR.

Components can be read out by irradiating the complete component or by addressing (electrically, opto-mechanically by x-y stage and e.g. fiber optics or opto-electrically, by coupling-in or receiving focussed light via deflectable micro-mirrors) individual fiber elements.

The sample molecule fields are addressable in the meaning that an association has been/is made between each sample molecule field or its geometric position for the component or the sample molecule species group carried by the sample molecule field. The association may be direct or indirect. In the latter case, for instance during production, first an association between sample molecules or sample molecule groups and (different) markings of the fields takes place. Basically this means that first a marking is associated to the sample molecules or sample molecule groups. After completion of the component, then a detection of the markings and and an association of the markings and consequently of the sample molecule species or sample molecule species groups to the spatial arrangement of the fields in the component is performed. A marking may for instance be made by integration or application of quantum spots at or in a field. Any other marking types, for instance color pigment coding, are also possible. The final association between sample molecule species/group to its spatial arrangement can be made by the manufacturer or also by the user. As a result, an exact spatial arrangement needs not be maintained during the original production process, rather after assembling the component a calibration is made by position-resolved detection of the markings .

Fiber elements may be pre and/or re-treated by coplanarly grinding fiber ends, grinding fiber ends so to form micro-lenses, metallically coating fibers for producing evanescent waves, roughening fibers, mirror-coating fibers at one end and thus detecting and measuring the emission of coupled-in light again at the same (not mirror-coated) end, as an option by the same optics device. The signal coupling or modulation can be amplified by introducing a modulating or reflecting dissolved substance in the fiber interspaces. This substance may however also exist there as a solid body.

All above statements apply in a corresponding manner to the method according to the invention and to the use according to the invention.

In the following, the invention will be explained in more detail, based on examples representing embodiments only.

### EXAMPLE 1

### Immobilization of Nucleic Acids on a Polymeric Endless Fiber.

An oligonucleotide of the sequence T(20)-AGT CTA ATC TGA TCT AGA was brought into a crosslink solution containing 1 M NaCl, 0.2 M MgCl₂, 0.3 M Tris, pH8, in a concentration of 10 nM. In a device a PS endless fiber of 80 μm diameter extruded in a conventional way is guided from a storage drum through a quartz glass tube having an interior diameter of 1 mm and a length of 1 m, the ends of said tube being open and bent upwardly, at one end of the tube an additional discharge port and at the other end an additional supply port being provided. The supply port is connected to a hose pump being connected to a storage container where the oligonucleotide solution is stored. The endless fiber is wound up at the exit end of the quartz glass tube on a collection drum driven by a stepper motor. The oligonucleotide solution is brought into the quartz glass tube, which is irradiated by a UV lamp arranged thereabove with a main emission at 254 nm. The speed of the collection drum is adjusted so that a duration of each region of the endless fiber in the oligonucleotide solution in the quartz glass tube of approx. 5 min is achieved. Simultaneously, the volume flow of the oligonucleotide solution through the quartz glass tube is adjusted such that within approx. 5 min a complete exchange of the oligonucleotide solution in the quartz glass tube takes place. Various endless fibers are thus coated in various quartz glass tube or reactors with various oligonucleotides. Alternatively, different sections of a single endless fiber, for instance each of 10 m length, can be coated with various oligonucleotides by that the oligonucleotide solution in the quartz tube is exchanged according to the desired section length and under consideration of the forward speed of the endless fiber through the quartz glass tube. In this way on a single endless fiber very many different specificities may be generated.

### EXAMPLE 2

### Immobilization of Nucleic Acids on a Glass Endless Fiber.

The endless fiber of glass is first guided through a silanization solution and amino-silanized. For the remaining process, the same is performed as in example 1, with the difference that the solution in the reactor contains an amino-specific chemical crosslinker, for instance EDC, and that the oligonucleotides are amino-modified. An irradiation is not necessary.

### EXAMPLE 3

### Jointing of Fiber Elements to a Component by Meander-Type Laying.

An endless fiber of 200 μm diameter and 1,000 m length being coated with different oligonucleotides at 1,000 equidistant different length sections (of 1 m length) is wound in an opposite sense over two opposite rows of 100 guide hooks each, so that the transition positions between the various length sections will come to lie at the guide hooks. The fiber is then at last laid in a meander-type manner, the various length sections being arranged in parallel to each other. The various length sections are then, possibly in the direction of the meander plane, brought to rest against each other, and the thus generated dense layer of length sections is then provided with a cover foil. There results a layer of 20 mm width and 1 m length. This is repeated 99 times with further length sections of the endless fiber or another endless fiber, the generated layers being stacked under respective interposition of a cover foil. A 100×100 pattern is produced, in a plane orthogonally to the longitudinal extension of the length sections. The cover foils are pulled out in the longitudinal direction, without displacement of the length sections, and a condensing pressure is applied. Then results a substantially square 100×100 pattern in a dense packing. Then, for the mechanical stabilization of the positions of the length sections with regard to each other, a supporting sleeve, for instance adapted as a ribbon, is positioned around the pattern, and the sections at the guide hooks are removed by two cuts in planes orthogonally to the longitudinal extensions of the length sections. By position-selective light coupling at a length section so produced and measurement of the obtained signal at the other end so obtained takes place a determination of the position of a length section and of the oligonucleotide associated hereto. Finally, in a direction orthogonally to the longitudinal extension, blocks of a height of 1 mm are cut off, nearly 1,000 biochips of an identical structure being obtained thereby.

### EXAMPLE 4

### Jointing of Fiber Elements by Weaving Technology.

Endless fibers, as used in example 3, are woven in according to a warp thread between weft threads in a meander-type manner. As weft threads serve fibers not coated with nucleic acids. There results so to speak an areal textile with length sections being parallel to each other. A plurality of such areal textiles are stacked and held on each other, the weft threads being then pulled out. During this or thereafter a condensation is performed, and there results a pattern according to example 3. In this embodiment of the invention, fibers provided with nucleic acids may also be used as weft threads (then these will be fibers of different endless fibers with only one oligonucleotide or oligonucleotide group each), and the warp threads will be formed of fibers not coated with oligonucleotides.

### EXAMPLE 5

### Jointing of Fiber Elements by Means of a Supporting Element.

An endless fiber according to example 3 is threaded to and fro through a plurality of thermoplastic perforated plates, the transitions of the various length sections coming to lie at the inversion points of the first and last perforated plates. Perforated plates arranged between the first and last perforated plates are fixed with regard to each other in the direction of the longitudinal extension of the length sections by for instance spacers, thus respective pairs of perforated plates being formed (between the pairs of perforated plates may also be arranged (shorter) spacers, but the perforated plates of adjacent pairs may also directly rest against each other). By heat action and/or mechanical pressure forces on the perforated plates in the directions of the main plane of the perforated plates, the length sections in each perforated plate are fixed therein so to speak in of a shrunk manner. Then cuts between the perforated plates of adjacent pairs are made. The outer faces of the perforated plates of the constructs so obtained are polished, so that the ends of the fiber elements are coplanar with the outer faces of the perforated plates. The perforated plates may be built up from an opaque material. The obtained construct may be provided with an opaque sleeve. The perforated plates may comprise supply and/or discharge openings for fluids, for instance analysates.

### EXAMPLE 6

### Execution of a Measurement with a Component According to the Invention.

A component according to the invention is contacted under hybridization conditions with an analysate containing a mixture of fluorescence-marked oligonucleotides. Some of the oligonucleotides of the analysate have complementarity to some oligonucleotides immobilized on the component. The analysate will automatically distribute within the component due to capillary forces, oligonucleotides of the analysate being complementary to immobilized oligonucleotides being bound by hybridization to the component or the respective fiber elements. Then follows a washing step with washing buffer, not hybridized oligonucleotides of the analysate being exchanged. Then a front face of the component is irradiated with a wavelength suitable for the excitation of the fluorescence dye. At the opposite side, a detection of signals is performed at the emission wavelength of the fluorescence dye, and that with a position resolution in the directions of the plane of the front face by means of a CCD element. The application of a scanner and/or photomultiplier is also possible. An evaluation is made under consideration of the positions of the fiber elements and of the signals respectively emitted therefrom.

### EXAMPLE 7

### Dynamic Addressing.

Different endless fibers are extruded from a polymeric granulate, with different quantum dots (with different wavelength characteristics) being mixed to various samples of granulate mass. In this way results a unique coding of the respectively produced endless fibers. The endless fibers are, as described above, coated with different sample molecule species or sample molecule species groups, an association between coding and sample molecule species/groups being made. Then, again as described above, from several different endless fibers is assembled a component. By the manufacturer or by the user, a spatial association of the local positions of the respective fiber elements with their respective coding is made by means of spectral analysis of the individual fiber elements. With the known association of the codings and the sample molecule species/groups then their association to spatial positions is made. As a result, during manufacture, the exact positioning of the fiber elements is not important.

### EXAMPLE 8

### Detection by Means of FRET.

A fiber element is provided with sample molecules that in case of contact with the specified target molecule create a co-operative interaction, for instance stacking. The co-operation partners are therein provided with one donor and one acceptor each for FRET. In case of a binding of the target molecule, a spatial proximity of the donor/acceptor pair takes place, which will lead to FRET in case of an optical contact with the excitation wavelength. This construction makes a marking of the target molecules, for instance with dyes, and a washing step unnecessary.

1. A component comprising a plurality of fiber elements and sample molecules of selected sample molecule species or selected sample molecule species groups that are immobilized on said fiber elements, to each fiber element being associated a specific sample molecule species or sample molecule species group,
characterized by
that the sample molecules are immobilized on outer surfaces of the fiber elements, and
that the fiber elements are fixed by means of a supporting element in a radial direction with regard to the fiber elements in an interspaced manner or are bundled together with linear contact.

2. A component according to claim 1, characterized by that the fiber elements are arranged parallely to each other as fiber element bundles.

3. A component according to claim 1 or 2, characterized by that the fiber elements are optical fiber elements.

4. A component according to one of claims 1 to 3, characterized by that the fiber elements are made from a polymeric material, preferably selected from the group consisting of “polyethylene (PE), polycarbonate (PC), polyvinyl chloride (PVC), polystyrene (PS), polyethylene terephthalate (PETP), polyethersulfone (PES), polyetherether ketone (PEEK), polyphenylene oxide (PPO), polyphenylene sulfide (PPS), polybutylene terephthalate (PBT), polyoxymethylene (POM), polysulfone (PSU), polyetherimide (PEI), polyamide (PA) and mixtures and copolymers of the monomers of such polymers”, in particular selected from the group consisting of “polycarbonate (PC), polyvinylchloride (PVC), polystyrene and mixtures and copolymers of the monomers of such polymers”.

5. A component according to one of claims 1 to 4, characterized by that the front faces of at least one end of the fiber elements, preferably of both ends, are optically contactable.

6. A component according to one of claims 1 to 5, characterized by that the fiber elements have a diameter in the range of 0.01 μm to 1,000 μm.

7. A component according to claim 1 to 6, characterized by that the fiber elements have a length in the range of 0.1 mm to 100 mm.

8. A component according to one of claims 1 to 7, characterized by that the fiber elements are packed in a density of 1 to 10⁷ fibers/cm², referred to a radial cross-section plane of the fiber elements.

9. A component according to one of claims 1 to 8, characterized by that the supporting element is arranged at one end of the fiber elements and structured so to enclose the ends of the fiber elements, the front faces of the enclosed fiber elements being directly or indirectly optically contactable.

10. A component according to one of claims 1 to 9, characterized by that one supporting element each is arranged at both ends of the fiber elements.

11. A component according to one of claims 1 to 8, characterized by that the supporting element is arranged between the two ends of the fiber elements, for instance centrally.

12. A component according to one of claims 1 to 11, characterized by that the supporting element is adapted as a perforated plate.

13. A component according to one of claims 1 to 12, characterized by that the supporting element is configured as a wound-up supporting ribbon.

14. A component according to one of claims 1 to 13, characterized by that the sample molecule species or sample molecule species is selected from the group consisting of “nucleic acids, DNA, RNA, PNA, aptamers, proteins, peptides, saccharides and mixtures of these sample molecules”.

15. A component according to one of claims 1 to 14, characterized by that each fiber element carries sample molecules of a respectively selected, different sample molecule species.

16. A component according to one of claims 1 to 15, characterized by that each fiber element carries sample molecules of a respectively selected, different sample molecule species group, the group elements of every sample molecule species group commonly binding under generation of co-operative effects to a defined target molecule.

17. A component according to claim 17, characterized by that each sample molecule species group contains two group elements.

18. A component according to one of claims 4 to 17, characterized by that the polymeric material does not carry any functional groups at its surface, that the sample molecules are nucleic acids, and that the nucleic acids from an aqueous fixing solution containing MgCl₂ and NaCl are bound under irradiation with UV light to the surface of the polymeric material.

19. A method for the production of a component according to one of claims 1 to 18, comprising the following steps:
a)at least one endless fiber is produced,
b)the endless fiber is guided through a fluid containing a selected sample molecule species or a selected sample molecule species group,
c) the sample molecules of the sample molecule species or sample molecule species group are immobilized on the endless fiber,
d) as an option the endless fiber is supplied to at least one washing step,
e) to the endless fiber is associated the sample molecule species or sample molecule species group immobilized on the fiber in step c)
f)from different endless fibers or from different regions of an endless fiber, one fiber element each is cut off, and the fiber elements of various endless fibers or sections are bundled and fixed.

20. The use of a component according to one of claims 1 to 18 in a method for the detection of target molecules, wherein optically contactable front faces of the fiber elements are optically connected to a detector being sensitive to optical radiation of the wavelength to be detected, and wherein signals of the detector are respectively associated to the fiber elements, comprising the following steps:
a) to the component is supplied a solution with prospective target molecules, under conditions at which target molecules bind to sample molecules,
b) simultaneously with step a) or subsequently thereto the component is irradiated with a primary radiation exciting a wavelength to be detected,
c)simultaneously with step b) or subsequently thereto a reading-out of the signals of the detector and processing and storage of the signals is performed.

21. The use of a component according to one of claims 1 to 18 in a method for the preparative processing of a solution containing a mixture of substances, the component carrying sample molecules binding to target molecules to be separated from the mixture of substances, comprising the following steps:
a) the solution is supplied to the component, the target molecules to be separated being bound to sample molecules and being thus immobilized,
b)then the solution made free from target molecules to be separated is led away from the component and possibly supplied to another processing step, for instance to a method according to one of claims 26 or 27.
